(19) United States
(12) Patent Application Publication
(10) Pub. No.: US 2011/0144400 A1
(43) Pub. Date: Jun. 16, 2011

(54) HIGHLY POROUS FOAM CERAMICS AS CATALYST CARRIERS FOR THE DEHYDROGENATION OF ALKANES

(75) Inventors: Muhammad Iqbal Mian, Dortmund (DE); Max Heinritz-Adrian, Munster (DE); Oliver Noll, Castrop-Rauxel (DE); Domenico Pavone, Bochum (DE); Sascha Wenzel, Bochum (DE)

(73) Assignee: UHDE GMBH, Dortmund (DE)

(21) Appl. No.: 13/057,937

(22) PCT Filed: Jul. 28, 2009
(86) PCT No.: PCT/EP09/05440

§ 371 Date: Feb. 7, 2011

(30) Foreign Application Priority Data
Aug. 7, 2008 (DE) 10 2008 036 724.9

(51) Int. Cl. C07C 5/333; B01J 29/06; B01J 23/10; B01J 23/26; B01J 23/58; B01J 23/835; B01J 23/62; B01J 23/02; B01J 21/02; B01J 23/06; B01J 21/06; B01J 23/14; B01J 21/10; B01J 37/02; B01J 37/08

(52) U.S. Cl. 585/440; 502/60; 502/64; 502/304; 502/306; 502/307; 502/308; 502/310; 502/320; 502/328; 502/329; 502/334; 502/336; 502/338; 502/339; 502/340; 502/341; 502/342; 502/343; 502/349; 502/350; 502/351; 502/352; 502/355; 585/600; 585/654

(57) ABSTRACT

The invention relates to a material which is suited as a carrier for catalysts in the dehydrogenation of alkanes and in the oxidative dehydrogenation of alkanes and which is made of an oxide ceramic foam and may contain combinations of the substances aluminium oxide, calcium oxide, silicon dioxide, tin oxide, zirconium dioxide, calcium aluminate, zinc aluminate, silicon carbide, and which is impregnated with one or several suitable catalytically active materials, by which the flow resistance of the catalyst decreases to a considerable degree and the accessibility of the catalytically active material improves significantly and the thermal and mechanical stability of the material increases. The invention also relates to a process for the manufacture of the material and a process for the dehydrogenation of alkanes by using the material according to the invention.

The invention relates to a material which is suited as a catalyst for the dehydrogenation of alkanes and which consists of a ceramic foam carrier impregnated with a catalytically active material. By means of the material according to the invention it is possible to run a process in which alkanes mixed with water vapour are dehydrogenated at elevated temperature to give hydrogen, alkenes and non-converted alkanes mixed with water vapour. By means of the material according to the invention it is also possible to run a process in which alkanes mixed with water vapour and oxygen undergo an oxidative dehydrogenation at elevated temperature to give alkenes, hydrogen, non-converted alkanes and reaction steam mixed with water vapour. The invention also relates to a process for the production of the material according to the invention.

The technically implemented dehydrogenation of alkanes involves the possibility of obtaining olefins on the basis of low-priced paraffins, which are more expensive because of the higher reactivity and for which there is an increased demand. The technical dehydrogenation of paraffins can be carried out in the presence of water vapour as a moderator gas, wherein the paraffin is dehydrogenated to give alkene and hydrogen. This process step is endothermal so that the reaction mixture cools down if no heat is supplied. This process step is therefore carried out as either adiabatic reaction in which a previously heated reaction mixture is passed through a heat-insulated reactor or as allothermal reaction in an externally heated tubular reactor.

It is possible to combine this process step with a subsequent oxidation step where the hydrogen obtained in the first step is combusted selectively. This produces heat on the one hand which can be used in the subsequent process steps. On the other hand the partial pressure of the hydrogen is decreased by the combustion of the hydrogen, by which the equilibrium of the dehydrogenation can be shifted in favour of the formation of alkenes. To achieve an improvement of the process implementation, the process steps of dehydrogenation and selective hydrogen combustion are usually implemented one after the other.

Allothermal dehydrogenation is carried out in a reforming reactor suited for this purpose. The reaction gas is heated indirectly by burners. Generally, the heat required by the reaction is not only compensated but the reaction gas leaves the reactor at a higher temperature. After the reaction, the product gas which still contains non-converted alkane is passed into the reactor for selective hydrogen combustion where it is re-heated by the combustion reaction and then recycled to the allothermal dehydrogenation process after separating the alkenes and by-products. The reaction implementation may comprise an arbitrary number and kind of intermediate process steps.

WO 2004039920 A2 describes a process for the production of non-saturated hydrocarbons wherein, in a first step, a hydrocarbon mixture containing preferably alkanes, which may also contain water vapour and does essentially not contain any oxygen, is passed through a first catalyst bed of standard dehydrogenation conditions in continuous operating mode, and subsequently water as well as water vapour and a gas containing oxygen are admixed to the reaction mixture obtained from the first step, and subsequently the reaction mixture obtained is passed in a second step through another catalyst bed for the oxidation of hydrogen and further dehydrogenation of hydrocarbons. This gives alkenes mixed with non-converted alkanes, hydrogen, by-products and water vapour. The alkene can be separated from the product mixture in suitable process steps.

For this process it is possible to use a catalyst which is suitable for both the dehydrogenation and the oxidative hydrogen combustion. A suitable catalyst is described in U.S. Pat. No. 5,151,401 . This catalyst is made by impregnating a carrier of a zinc aluminate compound with a chlorous platinum compound and fixing the platinum compound on the carrier in a calcining step. In a subsequent washing step, the carrier is then freed from chloride ions which could be set free in the process and have highly corrosive properties. To improve the properties of the carrier, the carrier may be mixed with the compounds zinc oxide, tin oxide, stearic acid and graphite.

The dehydrogenation process usually takes place at temperatures between 450 and 820° C. To allow that an adequate temperature be adjusted, water vapour is added to the process prior to the dehydrogenation and water vapour, hydrogen or a mixture of water vapour and hydrogen are added to the process prior to the oxidative hydrogen combustion. By adding water vapour it is also possible to reduce the amount of carbon depositing on the catalyst.

To allow that the through-passing gases reach adequately high flow velocities and to ensure an adequately high heat resistance of the catalyst, the carrier-supported catalyst is pressed into shaped bodies in a calcining or sintering process. Suitable shaped bodies are, for instance, cylindrical shaped bodies, pellets or spheres of an equivalent spherical diameter of 0.1 mm to 30 mm. The disadvantage of this geometry is, however, that it hampers the access of the reaction gas to the interior of the shaped body. Besides, the pressure loss, especially in the case of very dense catalyst fillings, continues to be significant. Loading of the catalyst shaped bodies into the reactor may in cases involve a high personnel and process expenditure due to the geometry of the shaped bodies. Last but not least it is also possible that the shaped bodies break which will adversely affect the flow property of the filling.

It is therefore the aim to find a catalyst geometry which ensures an adequately high flow velocity as well as an adequate accessibility of the catalyst at a pressure loss which is as low as possible. The catalyst should be of adequate mechanical and thermal stability even with increased flow velocity.

The invention achieves this aim by means of a foam ceramic which is composed of a specific combination of substances. The foam ceramic may be based on open-cell polyurethane (PUR) foams. Open-cell foam structures can be reached by eliminating (i.e. reticulating) the cell membranes in a subsequent process step. The substances are taken from the group of oxide ceramics such as aluminium oxide, calcium oxide, silicon dioxide, tin dioxide, zinc oxide and zinc aluminate or from non-oxide ceramics such as, for example, silicon carbide, boron nitride and the like. These substances may also be combined. By impregnating the PUR foam in a suspension of these substances, followed by drying and sintering, the foam ceramic is obtained which serves as carrier material. To establish the catalytic activity, the foam ceramic is impregnated with one or several suitable catalytically active materials. Typically this is metallic platinum. It is also possible to use different and additional catalytically active materials for impregnation if these are suitable for enabling the desired reaction.

Claim is especially laid upon a material for the catalytic conversion of gas mixtures which may contain C2 to C6 alkanes and hydrogen, oxygen or a mixture of hydrogen and oxygen, wherein mainly alkenes and hydrogen as well as additionally water vapour are obtained and

the material consists in ceramic foams which are made up of single components or of a mixture of oxide or non-oxide ceramic materials or of a mixture of oxide and non-oxide ceramic materials, and

the material is impregnated by at least one catalytically active substance to establish the catalytic activity.

The oxide ceramics are in particular the ceramic materials aluminium(III) oxide, calcium oxide, calcium aluminate, zirconium dioxide, magnesium oxide, silicon dioxide, tin dioxide, zinc dioxide or zinc aluminate. These materials may be used as single components or in a mixture. The non-oxide ceramics are in particular the ceramic materials silicon carbide or boron nitride. These materials may also be used as single components or in a mixture. Finally, mixtures of oxide and non-oxide materials can also be used for the manufacture of the carrier material.

To improve the carrier properties, the carrier material may contain an additional substance from the group of the substances chromium(III) oxide, iron(III) oxide, hafnium dioxide, magnesium dioxide, titanium dioxide, yttrium(III) oxide, calcium aluminate, cerium dioxide, scandium oxide or also zeolite. In addition, zirconium dioxide may also be used in combination with calcium oxide, cerium dioxide, magnesium oxide, yttrium(III) oxide, scandium oxide or ytterbium oxide as stabilisers.

A typical process for the manufacture of ceramic foams is taught by EP 260826 B1. In an exemplary manner, α-aluminium oxide as a suitable ceramic raw material is mixed with titanium dioxide as stabiliser and an aqueous solution of a polymer is added. After stirring this mixture, polyurethane foam pellets are added and the mixture is mixed. This is followed by the drying and sintering step which is carried out at a temperature of up to 1600° C. and makes the polyurethane foam matrix burn. The structure, a sintered ceramic foam, is obtained.

A possibility which is more simple is to pre-form the polyurethane foam into a suitable structure which typically follows the geometry of the application. The respective geometry may, for example, be a block or a cell bridge. This form is provided with a suspension of ceramic particles and with suitable auxiliary agents for sintering. These are thickeners, for example. The material is then subjected to a drying and sintering step at a temperature of up to 1600° C., in which the polyurethane foam burns and a structure of ceramic foam is obtained.

Macroporous ceramic materials as carriers for catalysts in dehydrogenation reactions for alkanes are known. U.S. Pat. No. 6,072,097 describes a macroporous ceramic material of α-aluminium oxide and other suitable oxide materials. The ceramic foam manufactured in this way is impregnated with platinum and tin or copper as catalytically active material. U.S. Pat. No. 4,088,607 describes a ceramic foam of zinc aluminate and a catalytically active material containing precious metals which is spread onto the foam. The catalyst manufactured in this way is well suited as an exhaust gas purification catalyst for automobiles, for example.

All known ceramic foams involve the disadvantage that their thermal and mechanical stabilities need to be yet improved. Many ceramic foams of adequate stability used as catalyst carriers are of disadvantageous influence on the catalytic properties of the impregnated material. This does not apply to the present combination of substances of which the carrier-supported material is manufactured.

It is possible to add further suitable auxiliary agents to the prefabricated material. This may be sawdust, for example. The auxiliary agents are incorporated into the material and burn in the sintering process so that pores are produced. Instead of sawdust any other material may be used that leaves pores after sintering and produces a ceramic foam.

This applies especially to catalysts which are suited for the dehydrogenation of alkanes or the selective hydrogen combustion. The substance combination according to the invention as a basis for a ceramic foam as carrier material for catalysts is also claimed by other applications. Examples are catalytic reforming processes, gas-phase oxidations or hydrogenations.

The carriers which are made of a ceramic foam of the material according to the invention are characterised by a high mechanical and also thermal stability and are of no negative influence on the impregnated catalytic material.

The manufacturing process allows exact adjustment of the porosity of the ceramic foam. In this way, it is optimally adaptable to the different flow properties in the respective application processes. The porosity of the foam can be characterised by the inner surface according to BET. Typical specific surfaces of the foams produced in the process according to the invention are up to 200 m²*g⁻¹. Typical pore densities of the foams produced in the process according to the invention are 5 to 150 PPI (PPI: “pores per linear inch”).

The catalytically active material on the carrier may be of any type desired. It will, in any case, be of a type that catalyses the requested reaction. Usually the catalytically active material is a platinum-bearing compound. It may be spread onto the carrier by, for example, impregnating with chlorous compounds. The chloride ions may be eluted from the ceramic foam in a subsequent washing step, as described in an exemplary manner in U.S. Pat. No. 5,151,401.

The material according to the invention is especially suited as a catalyst in the alkane dehydrogenation. Any type of alkane desired may be used as a starting compound. The material according to the invention is preferably used as a catalyst for the dehydrogenation of propane and n-butane to obtain propene and n-butene. Optional starting hydrocarbons, however, are also n-butene or ethyl benzene, in the case of which dehydrogenation will give butadiene or styrene, respectively. It is, of course, also possible to use alkane mixtures. The alkanes are preferably used with hydrogen, water vapour, oxygen or any mixture of these gases but may also be used in pure form.

The material according to the invention may be used as a catalyst for a dehydrogenation on standard dehydrogenation conditions. Typical dehydrogenation conditions are temperatures between 450° C. and 820° C. Especially preferred are temperatures between 500 and 650° C.

The material according to the invention in the form of a ceramic foam is suited as a carrier for catalytically active materials facilitating dehydrogenation or oxidative dehydrogenation of alkanes. By the process according to the invention it is possible to improve the flow resistance in reactors used to dehydrogenate alkanes to a considerable degree. The active use of the catalyst mass and the degree of pore utilisation can be improved significantly. The pore size and pore distribution can thus be adjusted more efficiently. The thermal and mechanical stability of the catalyst in alkane dehydrogenations can thus also be improved to a considerable extent. By the improved heat transfer in radial direction and the resulting lower radial temperature gradients within the tubular reactor it is possible to utilise the catalyst to an optimum degree.

1. Material for the catalytic dehydrogenation of gas mixtures which contain C2 to C6 alkanes and hydrogen, water vapour, oxygen or a any mixture of these gases, wherein mainly alkenes and hydrogen as well as additionally water vapour may be obtained,
characterised in that
the material consists in ceramic foams which are made up of single components or of a mixture of oxide or non-oxide ceramic materials or of a mixture of oxide and non-oxide ceramic materials, and
the material is impregnated by at least one catalytically active substance to establish the catalytic activity.

2. Material according to claim 1, characterised in that the oxide ceramics are the materials aluminium(III) oxide, calcium oxide, calcium aluminate, zirconium dioxide, magnesium oxide, silicon dioxide, tin dioxide, zinc oxide or zinc aluminate or a mixture of these materials.

3. Material according to claim 1, characterised in that the non-oxide ceramics are the materials silicon carbide or boron nitride or a mixture of these materials.

4. Material for the catalytic conversion of gas mixtures according to claim 1, characterised in that the material consists of a ceramic foam made of a mixture of the substances aluminium(III) oxide, calcium oxide, silicon dioxide, tin dioxide, zinc oxide, zinc aluminate, silicon carbide or boron nitride and additionally contains a substance from the group of materials chromium(III) oxide, iron(III) oxide, hafnium dioxide, magnesium oxide, titanium dioxide, yttrium(III) oxide, calcium aluminate, cerium dioxide, scandium oxide or zeolite.

5. Material for the catalytic conversion of gas mixtures according to claim 1, characterised in that the material consists of a ceramic foam made of a mixture of the substances aluminium(III) oxide, calcium oxide, silicon dioxide, tin dioxide, zinc oxide, zinc aluminate, silicon carbide or boron nitride and additionally contains a substance from the group of materials chromium(III) oxide, iron(III) oxide, hafnium dioxide, magnesium oxide, titanium dioxide, yttrium(III) oxide, calcium aluminate, cerium dioxide, scandium oxide or zeolite and zirconium dioxide in combination with calcium oxide, cerium dioxide, magnesium oxide, yttrium(III) oxide, scandium oxide or ytterbium oxide as a stabiliser.

6. Material for the catalytic conversion of gas mixtures containing alkanes according to any of the claims 1 to 5, characterised in that the foam ceramic is made of open-cell polyurethane foams or other open-porous plastic foams, the open-porous character of which may be achieved by any type of manufacturing process, wherein the foam is provided with a suspension of ceramic particles and suitable additives and the obtained foam undergoes sintering so that a foam ceramic is obtained, the manufacturing process of which allows exact adjustment of the form and the porosity and the foam ceramic is impregnated with a least one catalytically active material.

7. Process for the manufacture of a material according to any of the claims 1 to 6, characterised in that a ceramic precursor, which has been mixed with suitable additives as auxiliary agents in the production, is spread as suspension onto a prefabricated base material of polyurethane foam, after which the obtained material undergoes sintering at 1600° C., by which a ceramic foam is produced which is impregnated with a catalytically active material.

8. Process for the manufacture of a material according to claim 7, characterised in that finely distributed burnable materials are used as auxiliary agents which burn in the sintering process and leave pores in the ceramic foam.

9. Process for the manufacture of a material according to claim 8, characterised in that sawdust is used as an auxiliary agent.

10. Material for the catalytic conversion of gas mixtures containing alkanes according to any of the claims 1 to 9, characterised in that the specific pore surface of the foam ceramic is up to 200 m²*g⁻¹.

11. Material for the catalytic conversion of gas mixtures containing alkanes according to any of the claims 1 to 10, characterised in that the catalytically active material contains platinum, tin or chromium of mixtures thereof.

12. Process for the catalytic conversion of gas mixtures containing alkanes, characterised in that the alkanes are passed in a gas mixture, which may contain hydrogen, water vapour, oxygen or a mixture of these gases, via a catalyst which is supported by a porous foam ceramic carrier which is made of a mixture of the substances aluminium oxide, calcium oxide, silicon dioxide, tin dioxide, zirconium dioxide, calcium aluminate, zinc aluminate, silicon carbide or boron nitride and impregnated with a catalytically active material.

13. Process for the catalytic conversion of gas mixtures containing alkanes, characterised in that the alkanes are passed in a gas mixture, which may contain hydrogen, water vapour, oxygen or a mixture of these gases, via a catalyst, which is supported by a porous foam ceramic carrier which is made of a mixture of the substances aluminium oxide, calcium oxide, silicon dioxide, tin dioxide, zirconium dioxide, calcium aluminate, zinc aluminate, silicon carbide or boron nitride and additionally contains a substance from the group of materials chromium(III) oxide, iron(III) oxide, hafnium dioxide, magnesium oxide, titanium dioxide, yttrium(III) oxide, calcium aluminate, cerium dioxide, scandium oxide or zeolite and is impregnated with a catalytically active material.

14. Process for the catalytic conversion of gas mixtures containing alkanes, characterised in that the alkanes are passed in a gas mixture, which may contain hydrogen, water vapour, oxygen or a mixture of these gases, via a catalyst, which is supported by a porous foam ceramic carrier which is made of a mixture of the substances aluminium oxide, calcium oxide, silicon dioxide, tin dioxide, zirconium dioxide, calcium aluminate, zinc aluminate, silicon carbide or boron nitride and additionally contains a substance from the group of materials chromium(III) oxide, iron(III) oxide, hafnium dioxide, magnesium oxide, titanium dioxide, yttrium(III) oxide, calcium aluminate, cerium dioxide, scandium oxide or zeolite and zirconium dioxide in combination with calcium oxide, cerium dioxide, magnesium oxide, yttrium(III) oxide, scandium oxide or ytterbium oxide as a stabiliser and is impregnated with a catalytically active material.

15. Process for the catalytic dehydrogenation of gas mixtures containing alkanes according to any of the claims 12 to 14, characterised in that the dehydrogenation is carried out at a temperature between 450° C. and 820° C., the especially preferred temperature being between 500 and 650° C.

16. Process according to any of the claims 1 to 15, characterised in that the alkane to be dehydrogenated is n-propane or n-butane.

17. Process according to any of the claims 1 to 15 characterised in that the hydrocarbon to be dehydrogenated is n-butene or ethyl benzene.
